(19) United States
(12) Patent Application Publication
(10) Pub. No.: US 2025/0177249 A1
(43) Pub. Date: Jun. 5, 2025

(54) CLOSURE SYSTEM FOR A DRUG CONTAINER, AND DRUG CONTAINER COMPRISING A CLOSURE SYSTEM

(71) Applicant: INDUCTIO AG, Solothurn (CH)

(72) Inventor: Horst KOLLER, Solothurn (CH)

(73) Assignee: INDUCTIO AG, Solothurn (CH)

(21) Appl. No.: 18/843,699

(22) PCT Filed: Mar. 3, 2023
(86) PCT No.: PCT/EP2023/055418

§ 371 Date: Sep. 3, 2024

(30) Foreign Application Priority Data
Mar. 3, 2022 (EP) 22160078.6

(51) Int. Cl. A61J 1/14; B65B 3/00; B65D 51/00

(52) U.S. Cl. CPC A61J 1/1425; A61J 1/1406; B65B 3/003; B65D 51/002

(57) ABSTRACT

A closure system for a medication container, the interior of which is accessible via a container opening designed in the manner of a bottle mouth, comprising a bump cap with an annular lid having a central opening, on the outer circumference of which is arranged a number of latching elements which can be brought into engagement with an outer bead mounted circumferentially on the container opening, and a one-piece closure plug with a closure body which completely fills the central opening of the annular lid and can be brought into latching engagement therewith, is intended to enable continuous compliance with even high sterility requirements during handling in a simple and reliable manner. For this purpose, the closure body is designed as a duckbill valve.

### CROSS-REFERENCE TO RELATED APPLICATION

This application is a national phase of International Application No. PCT/EP2023/055418, filed Mar. 3, 2023, which is hereby incorporated by reference in its entirety.

### FIELD

This disclosure relates to a closure system for a medication container, and more particularly to a closure for a medication container, the interior of which is accessible via a mouth region designed in the manner of a bottle mouth, the closure system comprising, in addition to a push-on cap which can be pushed onto the mouth region and is provided with a sealing element, a locking ring which can be pushed onto the push-on cap and, in a position pushed completely onto the push-on cap, can be fixed to the push-on cap in a latchable manner by means of a number of snap-on ribs. It further relates to a medication container with such a closure system and to a use of the closure system.

### BACKGROUND

Medicines or pharmaceuticals, such as vaccines, are usually provided in active ingredient or medicine containers, also known as containers or vials. Such a medication container is usually designed in the form of a vial and comprises an interior in which the medication or active ingredient is stored and which is accessible via a container opening designed in the form of a bottle mouth. From such a container, the active ingredient is then transferred via suitable transfer systems for the actual administration to suitable systems such as a syringe or an intravenous line that provides fluid access to the patient's circulation.

The filling of such medication containers with active ingredient is usually automated in suitably designed filling systems in which a large number of medication containers can be processed and filled simultaneously. The medication containers are combined in batches of, for example, 100 units per batch and filled together (so-called “nesting”). Accordingly, the medication containers should be designed to be suitable for such automated handling, even in large quantities; this aspect is particularly important for active ingredients that need to be provided in large quantities in a short time, such as vaccines.

For various reasons, for example because of particularly high-quality or possibly also toxic or otherwise harmful active ingredients, it may also be desirable or even necessary to minimize or completely avoid unintentional loss of active ingredients both during filling and during subsequent handling, storage and removal of active ingredients. For proper functioning of such systems with the desired avoidance of unintentional loss of active ingredients, the appropriate design of the medication containers may be necessary, among other things. Usually, the active ingredient or medication containers are provided with suitable closure systems for this purpose, in which a closure plug closes the container opening. This sealing plug can then be pierced to remove the medication, for example using a hollow needle, which can then be used to suck the medication out of the container. To secure the stopper, a pop-up cap with a ring lid with a central opening can be provided, which can be attached to the “bottle mouth” with the container opening. The stopper is then fitted centrally in this ring lid.

Such a system of the above-mentioned type, which satisfies the above-mentioned requirements of automated handling on the one hand and a particularly high degree of impermeability on the other to a particularly extensive extent, is known, for example, from the European patent application number 20209611, which has not been pre-published.

However, it has been found that, in addition to the above requirements, sterility can also play a special role in the overall process, particularly with regard to the production or filling of large quantities of active ingredient. Usually, drug containers or the components of which they are composed are suitably sterilized during or immediately after their manufacture and then sterile-packed. In the sterile packaging, they are then sent for further use, such as filling. The filling itself should then usually also be carried out under sterile or aseptic conditions, so that contamination or impurity of the active ingredient filled in is reliably excluded. However, such continuous compliance with the sterility chain can be associated with increased effort, especially if sterility cannot be maintained in an intermediate step when handling the medication containers and the material therefore has to be sterilized again.

### SUMMARY

This disclosure is therefore based on the task of providing a closure system of the above-mentioned type for a medication container, with which, even while complying with high tightness criteria, compliance with sterile boundary conditions during handling of the system, in particular during filling, is particularly favored. Furthermore, a medication container with such a closure system and a particularly favorable use of such a closure system are to be disclosed.

With regard to the closure system, this task is solved in accordance with this disclosure in that the closure body of the closure plug is designed as a duckbill valve.

Duckbill valves, also known as duckbill check valves, are used in fluid transfer systems where the valve opens or closes depending on the direction of flow of the medium or fluid. This can prevent unwanted backflow of the medium in standard applications. For this purpose, such a duckbill valve comprises a number of valve flanks, usually two, which converge towards each other and lie against each other along a contact line in the “unloaded” resting state. If an overpressure is applied to the valve flanks “in the direction of flow” in a fluid channel, the valve flanks are forced apart due to the elastic properties of the material forming the valve body and open up a flow opening for the medium. This allows the medium to flow in the opening direction. If, on the other hand, overpressure is applied in the opposite direction, i.e. “in the return flow direction”, the valve flanks are pressed against each other in the area of the contact line and thus close the valve; a return flow is then not possible or at least only possible to a very limited extent.

Advantageous embodiments of the invention are the subject of the subclaims.

This disclosure is based on the consideration that, in order to particularly favor compliance with sterility conditions, possible sources that could be used to introduce undesirable impurities or contaminants into the interior of the container should be eliminated as far as possible. In the present case, the phase in which the container is filled with the medication or active ingredient is regarded as such a possible source, as open access to the inside of the container must be provided during this phase. During filling, in known systems a closure membrane provided as part of the closure system is usually punctured using a suitable needle in order to create the desired access to the container interior. However, this piercing in particular could lead to an undesired entry of impurities into the interior, as particles or other impurities adhering to the outer surface of the sealing membrane can be introduced into the interior through the tip of the needle.

Instead, the design of the closure body as a duckbill valve ensures that the needle system merely displaces or displaces the valve flanks when it is inserted into the interior of the container, without the need to pierce a diaphragm or the like. When the needle system is inserted into the beak area of the valve, the flank surfaces forming the valve and running towards each other are pushed apart, whereby the valve opens. When the needle system is pulled out after the medication or active ingredient has been filled in, the flank surfaces then close again to a large extent automatically due to the elastic restoring forces in the valve body.

A particularly high sealing effect in general can also be achieved by advantageously forming a radial sealing element with a cross-section adapted to the clear width of the container opening and slightly larger than the clear width of the container opening with regard to the deformability of the material of the sealing plug. This means that the sealing effect that can be achieved by the sealing plug can be further increased by utilizing radial force components, i.e. contact forces that press the sealing plug against the inside of the container opening in the radial direction, in addition to the axial force components that are normally used. In order to take this into account, the closure plug is advantageously designed in its area protruding into the container opening with regard to the shape and dimensions of its cross-section in such a way that, taking into account the deformability of its material, a flat pressing or contact pressure effect is created on the inner wall of the container mouth.

For particularly safe and reliable handling, the closure system should be specially secured once it has been attached to the medication container. To this end, it advantageously comprises a further component in the form of a locking ring which can be slid onto the pop-up cap and which can be fixed to the pop-up cap by means of a snap-on edge formed on the inside and arranged around the end. Once the bump cap has been fitted, in which the latching elements arranged on its outer circumference are brought into engagement, i.e. “snapped on” or latched, with the outer bead arranged around the container opening when it is pushed onto the container mouth, the retaining ring can be pushed onto the bump cap. It encloses the latching elements in a ring shape from the outside and thus fixes them in the latched position; it is then no longer possible to move outwards and thus release the latch.

In order to enable a particularly high suitability also for automated filling processes, the snap-on rib or each snap-on rib provided in the position completely slid onto the bulging cap for fixing the retaining ring is advantageously guided in a corresponding guide slot when the inner lateral surface of the retaining ring is moved relative to an outer lateral surface of the bulging cap corresponding to this. This ensures particularly reliable guidance of the pre-assembled components relative to each other, even for high cycle or throughput rates with the correspondingly high processing speeds when pre-assembling the medication containers. In particular, this makes it possible to eliminate potential sources of interference during automated processing, for example due to tilting or tilting of the components, incorrect positioning or similar. The desired reliable guidance of the pre-assembled components relative to each other can be achieved in a particularly simple way by forming a guide pairing for the components using components that are usually already present, in this case the respective snap ribs. To form such a guide pairing, a corresponding guide slot, for example in the form of a groove, should be assigned to the respective snap rib in the other component.

Due to its valve function, the closure body of the container interior, which is designed as a valve, might possibly not meet the highest tightness requirements per se. In order to take this into account and to ensure that the closure system described above also meets the highest tightness requirements, a further sealing element in the form of a pierceable sealing membrane is assigned to the closure body of the closure plug in the form of a functional supplement in a design that is regarded as independently inventive. For this purpose, a retaining collar for an associated sealing cap is formed around the central opening of the bump cap, so that the sealing cap can be easily attached to the bump cap by means of this retaining collar. In a further advantageous embodiment, the sealing cap has a retaining ring on which the pierceable sealing membrane, which rests on the closure body in the assembled state, is arranged.

Advantageously, the closure system is provided with a tamper-evident closure for the medication container in the form of a disposable closure. This disposable closure, which can for example comprise a tear-off or sealed sealing lid, allows easy and reliable identification of whether the container has already been used for liquid transfer or not, and thus makes it easier to determine whether the container has already been “opened” and should therefore preferably be used for further liquid withdrawal until it has been completely emptied and should therefore be disposed of. Advantageously, a sealing plate that can be torn off the retaining ring is arranged on the retaining ring of the closure cap to form a tamper-evident closure.

The sealing cap is attached to the bulging cap in the form of a bayonet lock for particularly easy installation and, in particular, for temporary removal from the bulging cap. In this context, bayonet lock means in particular that the sealing cap can be fastened to the bulge cap by means of a more or less far-reaching twist. For this purpose, the retaining collar of the pop-up cap is advantageously provided on the outside with a number of guide slots, in each of which a corresponding locking pin arranged on the inside of the retaining ring of the closure cap is guided.

A medication container, the interior of which is accessible via a container opening designed in the manner of a bottle mouth, closed with a closure system of the type described above, is also regarded as independently inventive.

The use of a closure system of the aforementioned type for a medication container is also regarded as independently inventive.

A filling device for a medicine container is also regarded as independently inventive, which comprises an injector unit connected to a storage container for a substance to be filled, which has a filling needle guided in an outer protective tube.

The advantages achieved with this disclosure include in particular in the fact that, by designing the closure body of the closure plug as a duckbill valve, the medication container can be filled through the closure plug by means of a suitable filling needle system, in that the needle system merely displaces or displaces the components of the valve without the need to pierce a membrane or the like. When the needle system is inserted into the beak area of the valve, the flank surfaces forming the valve and running towards each other are pushed apart, whereby the valve opens. When the needle system is pulled out after the medication or active ingredient has been filled in, the flank surfaces then close again largely automatically due to the elastic restoring forces in the valve body. Overall, this minimizes the risk of impurities, external particles or the like being introduced, so that filling is particularly reliable with such a closure system, even under high cleanliness requirements, especially under sterile conditions.

### BRIEF DESCRIPTION OF THE DRAWINGS

Embodiments of this disclosure are explained in more detail with reference to a drawings, which show:

FIG. 1 illustrates a medication container closed with a closure system,

FIG. 2 is an exploded perspective view of the medication container according to FIG. 1 with associated closure system,

FIG. 3 is an exploded view of the medication container according to FIG. 1 with associated closure system in longitudinal section,

FIG. 4 illustrates the closure system of the medication container according to FIG. 1 in perspective section in exploded view,

FIG. 5 illustrates the closure system of the medication container according to FIG. 1 in perspective section in pre-assembled condition,

FIG. 6 illustrates a stopper of the medication container according to FIG. 1 in different perspective views (FIG. 6a, 6b) and in longitudinal section (FIG. 6c),

FIG. 7 illustrates an alternative embodiment of a stopper of the medication container according to FIG. 1 in different perspective views (FIGS. 7a, 7b) and in longitudinal section (FIG. 7c),

FIG. 8 illustrates a sealing plate of the closure system according to FIG. 1 in perspective view from below,

FIG. 9 illustrates the pop-up cap of the closure system of the medication container according to FIG. 1,

FIG. 10 illustrates the pop-up cap as shown in FIG. 9 with inserted sealing plug and pushed-on locking ring,

FIG. 11 illustrates a sequence of steps in attaching the closure system to the medication container shown in FIG. 1 for pre-assembly,

FIG. 12 illustrates a sequence of steps in the filling of the medication container pre-assembled with the closure system according to FIG. 1 with an active ingredient,

FIG. 13 illustrates an enlarged sectional view of a sequence of steps in the insertion of a filling needle system into the medication container according to FIG. 1,

FIG. 14 is a top view of the expanding cap as shown in FIG. 9 with the filling needle system inserted,

FIG. 15 illustrates the mouth area of the duckbill valve with inserted filling needle in two different variants in cross-section, and

FIG. 16 is, schematically, a flow chart for the automated filling of a large number of medication containers with active ingredient as shown in FIG. 1.

Identical parts are marked with the same reference signs in all figures.

### DETAILED DESCRIPTION

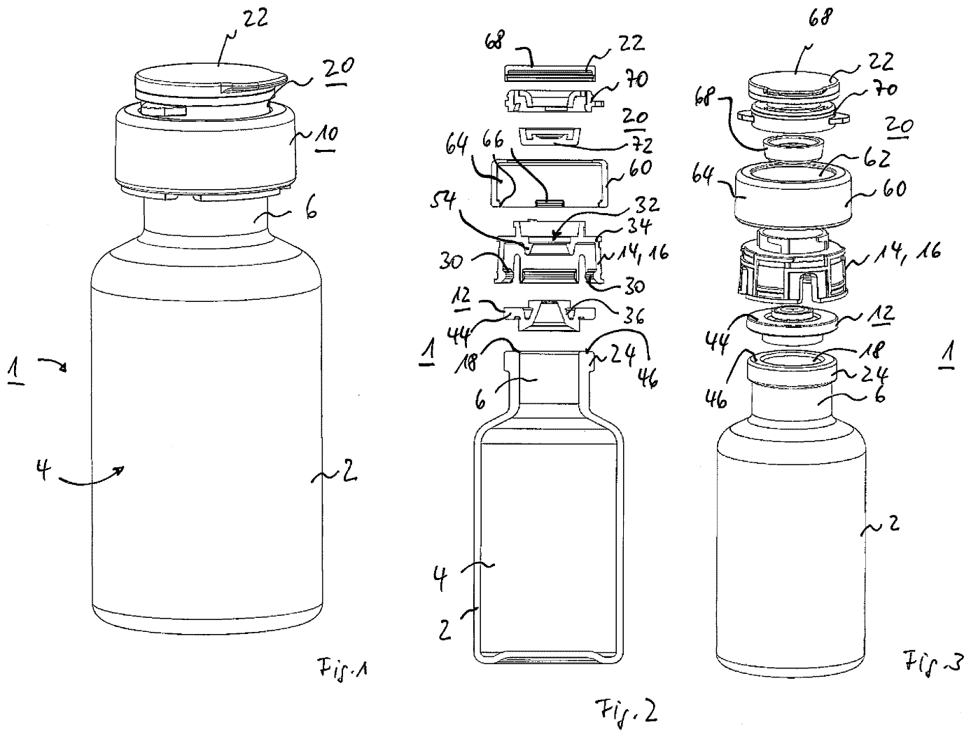

The medication container 1 as shown in FIG. 1, also known as a container or vial, is designed in the form of a small bottle. It comprises an interior 4 enclosed by a container wall 2, in which the medication or active ingredient is held. In the embodiment shown, the container wall 2 is made of a suitably selected plastic with or without a barrier layer. A “medical grade” plastic is particularly preferred, such as COP variants 690R®, 790R®, COC variants Topas® 8007S-04, 6013S-04, 6015S-04. The plastic is particularly preferably selected with regard to the criteria of transparency, resistance to breakage, little to no interaction with the intended medication, medical grade, in particular usable as a glass substitute, individually or in combination with each other. The interior 4 is accessible via a container opening 6 designed in the manner of a bottle mouth.

The medication container 1 is designed to be particularly suitable for automated filling with active ingredient under aseptic or sterile conditions. In particular, the components of the medication container 1 should be completely sterilized or sterilized as required after manufacture and then further treated, stored or otherwise used under sterile conditions without the need for subsequent complete sterilization. The filling of the container with active ingredient is considered to be a weak point in this respect, as the container interior 4 must be made accessible for this purpose and thus an entry of impurities or contaminants would be conceivable. In order to meet the associated requirements with regard to the desired continuous maintenance of sterility in a particularly extensive manner, the medication container 1 is equipped with a closure system 10 that closes the container opening 6, with which, on the one hand, the risk of unwanted impurities entering the container can be kept particularly low, especially during filling, while, on the other hand, particularly effective protection against tampering should also be provided.

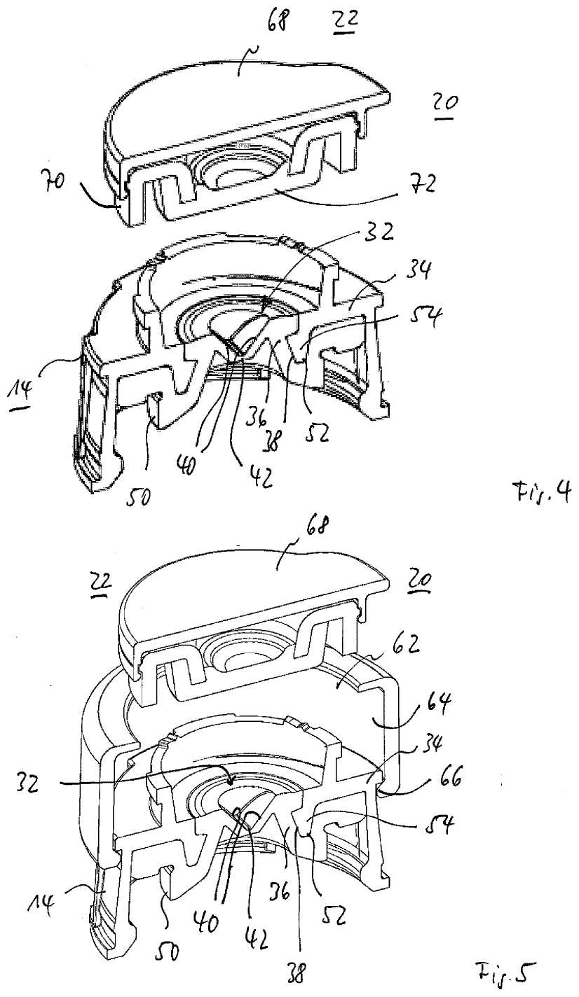

The closure system 10 comprises, as can be clearly seen from the longitudinal sectional view in FIG. 2 and the perspective view in FIG. 3, each in exploded view, the closure system 10 comprises as essential elements a closure plug 12, with which the container opening 6 of the container interior 4 is closed, a fixing cap 16 designed as a bump cap 14, with which the closure plug 12 is fixed to the mouth opening 18 of the medication container 1, and a closure cap 20 with a tamper-evident closure 22 attached thereto. Their preferred embodiments and respective functions are described in more detail below.

The container wall 2 of the medication container 1 is provided in the region of the container opening 6 with a number of, in the embodiment example one, circumferentially attached outer bead 24 as a fastening element for the closure system 10. In the embodiment example, as can be clearly seen from the illustration in FIG. 2, the outer bead 24 is completely circumferential and positioned in the vicinity of the mouth opening 18 of the container opening 6. In other embodiments, the outer bead 24 could be completely or partially segmented, i.e. comprise several segments which follow one another in the rotational direction and are positioned at a distance from one another, forming gaps between them.

Essential components of the closure system 10 are the sealing element designed as a closure plug 12 for closing the container opening 6 and the fixing cap 16, with which the closure plug can be firmly attached to the container opening 6. Adapted to the outer bead 24, the fixing cap 16 is designed as a pop-up cap 14, on the outer circumference of which a number of snap-in hooks or latching elements 30 that can be brought into engagement with the respective outer bead 24 are arranged. When attaching the pop-up cap 14, it can thus be pushed or bounced onto the container opening 6, with the latching elements 30 first being bent outwards through the respective outer bead 24 and then, after further pushing on, engaging behind the outer bead 24 and latching with it in the manner of a snap connection.

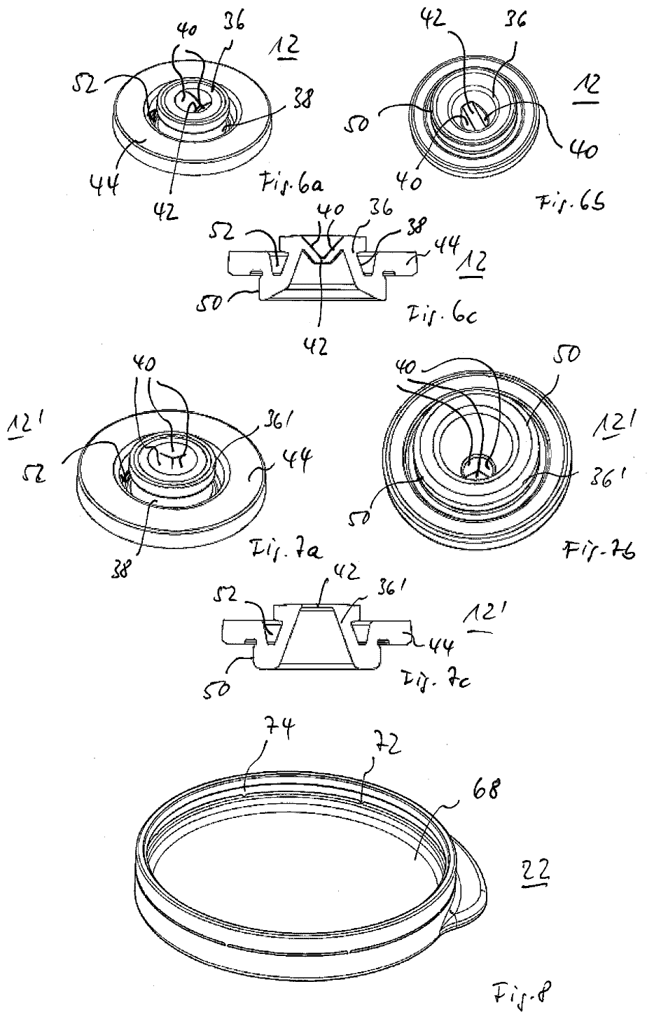

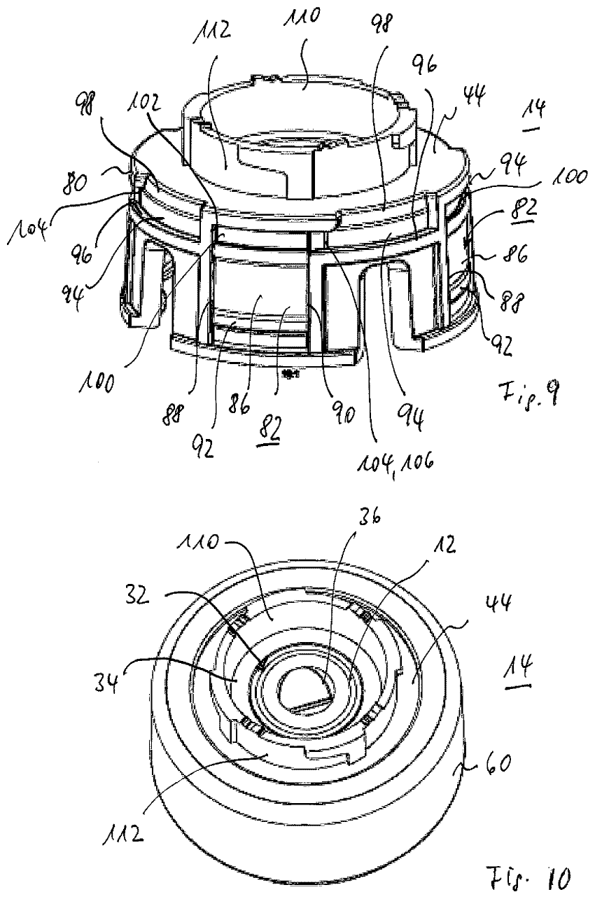

The snap-on cap 14 comprises an annular lid 34 having a central opening 32. The closure plug 12, which is designed as a single piece and is shown in the perspective sections in FIGS. 4 and 5 in the assembled state and enlarged in various views in FIG. 6, on the other hand, comprises a central closure body 36 in the manner of a basic element, which can be brought into engagement with the annular lid 34 in a latching manner at its “upper” end, which in the assembled state faces away from the container interior 4, in the opening 32. For this purpose, the closure body 36 is provided in its connection area with the annular lid 34 with a circumferential groove 38 forming an undercut. The closure plug 12, which is in itself a single piece, is manufactured from a suitable and also comparatively soft and easily deformable material, in the embodiment example from rubber or from TPE, preferably “medical grade”, also with regard to the desired sealing purposes. By utilizing these material properties, in particular the deformability, the closure plug 12 can be approximately firmly connected to the bump cap 14 by inserting the closure body 36 into the opening 32 in the annular lid 34 and the circumferential edge of the opening 32 then engages in the groove 38 and thus fixes the closure plug 12 to the bump cap 14.

The closure plug 12 is specifically designed to minimize the risk of contamination, external particles or the like entering the medication container 1 during media transfer into the medication container 1, i.e. during filling, and also out of the medication container, i.e. during removal of active ingredient from the container interior 4. This is intended in particular to ensure that the filling of the container interior 4 and also the removal of active ingredient can be ensured even under high cleanliness requirements, in particular under sterility conditions. To make this possible, the closure body 36 of the closure plug 12 is designed as a so-called duckbill valve.

Such a duckbill valve, also known as a duckbill check valve, is widely used in fluid transfer systems in which the valve opens or closes depending on the direction of flow of the medium or fluid. This prevents unwanted backflow of the medium in common applications. For this purpose, such a duckbill valve comprises a number of valve flanks 40, usually two, which run towards each other and lie against each other along a contact line 42 in the “unloaded” rest state. If the valve flanks are now subjected to an overpressure “in the direction of flow” in a fluid channel, the valve flanks 40 are forced apart due to the elastic properties of the material forming the valve body and open up a flow opening for the medium. This allows the medium to flow in the opening direction. If, on the other hand, overpressure is applied in the opposite direction, i.e. “in the return flow direction”, the valve flanks 40 are pressed together in the area of the contact line 42 and thus close the valve; a return flow is then not possible or at least only possible to a very limited extent.

The use of such a duckbill valve in the closure body 36 of the closure plug 12 now has the effect according to the invention that a media transfer, i.e. the filling of the medication container 1 or the removal of active substance from the medication container 1, can take place through the closure plug 12 by means of a respectively suitable needle system, for example an injection needle or a spike system for removal, in that the filling needle system merely has to displace or shift the valve flanks 40 of the valve without a membrane or the like having to be pierced in the process. When the filling needle system is inserted into the beak area of the valve, the valve flanks 40, which form the actual valve and run towards each other, are pushed apart, whereby the valve opens. When the filling needle system is pulled out after the medication or active ingredient has been filled in or removed by an injection needle, the valve flanks 40 then close again to a large extent automatically due to the elastic restoring forces in the valve body.

In the illustrations according to FIG. 6, the closure plug 12 is shown in an embodiment with a duckbill valve in the “usual” design, namely with two valve flanks 40. Alternatively, however, the duckbill valve can also be designed with more than two valve flanks 40, which then abut against each other in the idle state, forming several contact lines 42. As an example, FIG. 7 shows an alternatively closure plug 12′, the closure body 36′ of which is designed as a duckbill valve with three valve flanks 40, which abut against each other at three contact lines 42.

In an advantageous manner, the closure plug 12 designed as a sealing element contributes to sealing the container opening 6 in two ways. On the one hand, the closure plug 12 has a sealing plate 44 formed on the closure body 36, 36′ and running radially around it. This achieves a sealing effect, which is quite comparable with known systems, in that in the assembled system the sealing plate 44, which is suitably adapted in its dimensions, in particular its outer diameter, to the mouth edge 46 of the container opening 6, is pressed onto the mouth edge 46 by means of the bump cap 14, which can be latched onto the mouth edge 46. As a result of this axial force effect in relation to the longitudinal axis of the container opening, the sealing plate 44 can already develop a sealing effect due to the deformability of the material. In addition, however, the provision of radial force components, i.e. contact forces which press the sealing element in the radial direction against the inside of the container wall 2 in the region of its mouth, is also provided in the present case for a particularly increased sealing effect overall.

For this purpose, a radial sealing element 50 is formed on the closure body 36 “below” the sealing plate 44, i.e. on its side facing the container interior 4 in the assembled state. The cross-sectional shape of this is adapted to the cross-sectional shape of the container opening 6 in the mouth area (in the embodiment example, both are round). In terms of its dimensions, it is also adapted to the clear width 1 of the container opening 6 and is slightly larger than the clear width 1 of the container opening 6 with regard to the deformability of the material of the closure plug 12. As a result, when the radial sealing element 50 is inserted into the container opening 6, taking into account the deformability of its material, a flat pressing or contact pressure effect is created on the inner wall of the container in the area of the container opening. With regard to common standards and usual norms for such components, the container opening can be suitably selected and dimensioned; for example, its clear width can be suitably adapted to the standard dimension “13 neck” (corresponds to an outer diameter of the container opening of 13 mm), to the standard dimension “20 neck” (corresponds to an outer diameter of the container opening of 20 mm) or adapted to special variants for the neck geometry for the inner diameter.

The closure plug 12 is advantageously designed for an even further improved sealing effect in the radial direction. For this purpose, the shape is selected such that the central region of the closure plug 12, 12′ forming the closure body 36, 36′ is surrounded by a circumferential groove-like or trench-like recess 52 extending deep into the sealing plate 44. The recess 52 can also completely penetrate the material thickness of the sealing plate 44, so that the closure plug 12, 12′ is multi-component in this embodiment. Adapted to this, as can be seen in FIGS. 4 and 5, the bump cap 14 has a reinforcing ring 54 formed on the underside of the annular lid 34 and surrounding the opening 32. During assembly of the two components, this reinforcing ring 54 is inserted into the recess 52 of the closure plug 12. The dimensions are matched in such a way that the reinforcing ring 54 gives the radial sealing element 50 of the closure plug 12 increased strength and rigidity towards the outside, i.e. in the radial direction, and thus further improves the radial seal. In particular, the appropriately selected dimensions of the reinforcing ring 54 can slightly deform the radial sealing element 50 more or less outwards, thereby generating an additional contact pressure force in the radial direction on the inner wall of the medication container 1 in the region of the container opening 6.

In the embodiment example, the bump cap 14 is made of a suitably selected plastic, namely polypropylene (PP), a polyolefin, cyclo-olefin copolymer (COC), cyclo-olefin polymer (COP) or polycarbonate.

As a further component, as is again clear from the illustrations in FIGS. 2, 3 and 5, the closure system 10 comprises a retaining ring 60 which can be pushed onto the bump cap 14. This is designed as a ring structure with a cylindrical outer surface 64, leaving a sufficiently large central opening 62; this can be pushed onto the bump cap 14 from the outside in an encircling manner after the bump cap 14 has been popped up and latched with the outer beads 24. The retaining ring 60 thus fixes the latching elements 30 radially via its cylindrical outer surface 64 so that they can no longer move outwards. As a result, the latching of the bump cap 14 with the outer bead 24 can no longer be easily released and is therefore fixed. For its part, the retaining ring 60 has a number of snap ribs 66 formed on the inside of its cylindrical outer surface and positioned at the ends, by means of which it can be fixed in a latching manner to the bump cap 14.

Furthermore, the medication container 1 closed with the closure system 10 has the tamper-evident closure 22 as a component. This is intended to ensure, in the manner of a disposable closure, that the user can easily and reliably determine whether the medication container 1 has already been used for liquid transfer or not, i.e. whether active ingredient has already been removed or not. This makes it easier to determine whether the container has already been “opened” and should therefore preferably be used for further liquid removal until it is completely empty and should therefore be disposed of. The tamper-evident closure 22 is designed as a sealing plate 68 formed on the closure cap 20, as can be seen in the perspective section according to FIG. 5.

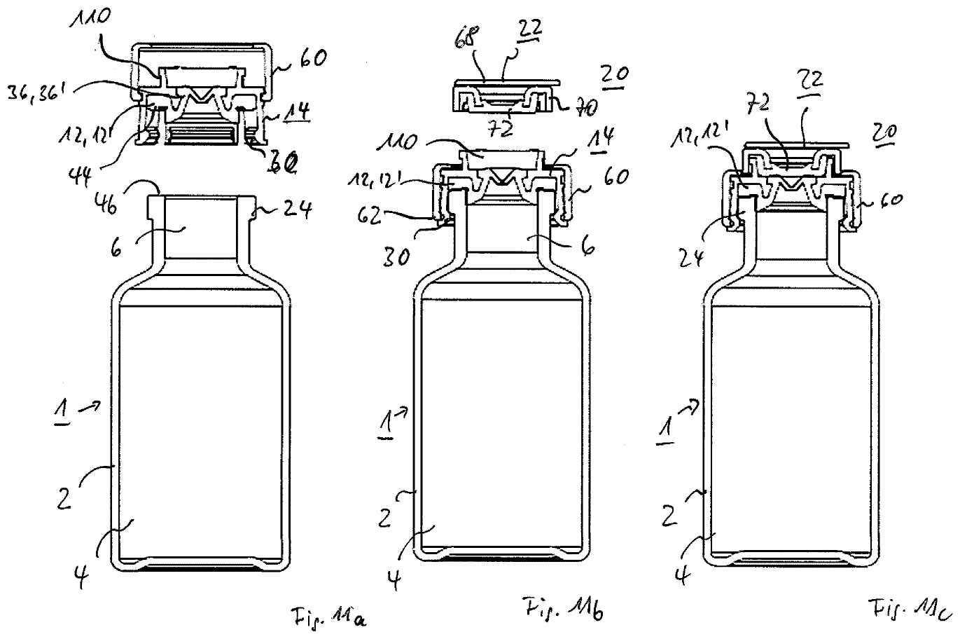

The closure cap 20 is provided in an independently inventive embodiment as a functional supplement to the closure body 36, 36′ of the closure plug 12, 12′, which is designed as a duckbill valve. Since the closure plug 12, 12′ could possibly not have a completely hermetically sealing effect precisely because of its design as a valve function, especially when the filled medication container 1 is stored for a longer period of time, the closure cap 20 is designed as a supplementary sealing means. For this purpose, it comprises a sealing membrane 72 attached to a retaining ring 70, which rests directly on the closure body 36, 36′ when the system is assembled and thus seals it off from the outside. The sealing membrane 72 is dimensioned and positioned in such a way that, in the assembled state, it completely covers the central opening 32 of the annular lid 34 and thus the exposed surface of the closure plug 12 accessible above it. To form the tamper-evident closure 22, the sealing plate 68 is also attached to the retaining ring 70 so that it can be torn off. To access the inside of the medication container 1, i.e. to remove the active ingredient, the sealing plate 68 must first be removed and then the sealing membrane 72 must be pierced before a corresponding needle system can be inserted through the duckbill valve underneath. For clarification, the sealing plate 68 is shown in FIG. 8 in perspective view from below, whereby some of the fixing points 74 are also recognizable, at which the sealing plate 68 is attached to the retaining ring 70 so that it can be torn off.

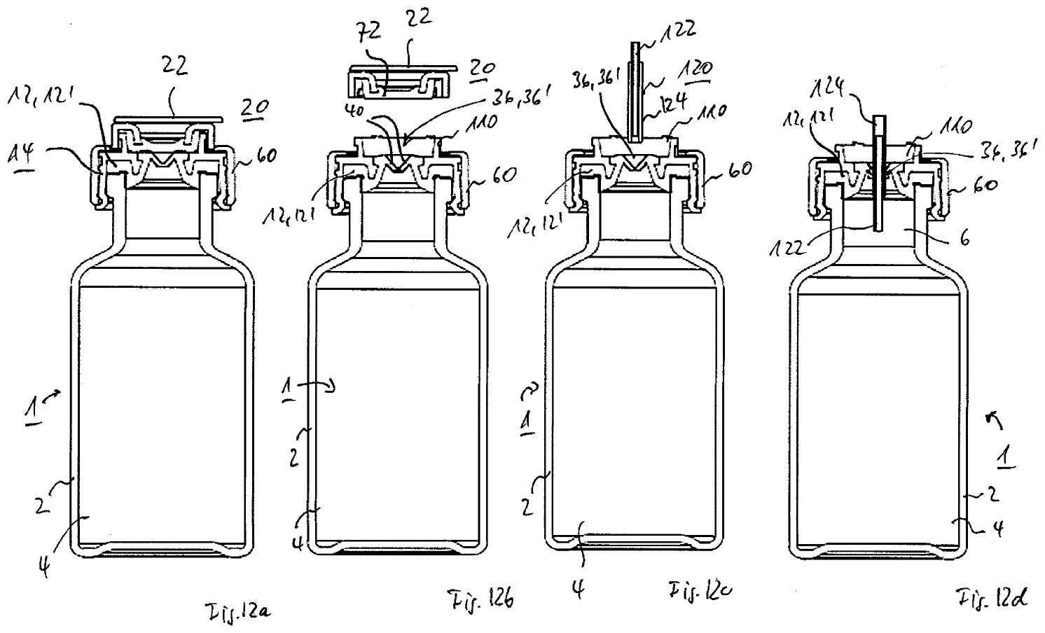

The closure system 10 of the medication container 1 is designed in a configuration that is considered to be inventive in its own right for particularly stable pre-assembly of the retaining ring 60 on the bump cap 14, so that the pre-assembled system is also particularly suitable for subsequent process steps involving high stresses, for example in the context of automated filling or packaging processes. For this purpose, the snap ribs 66 arranged on the inside of the retaining ring 60 are also used in the manner of an additional function to form a guide pairing which, as shown in the perspective view of the snap-on cap 14 in FIG. 9, also comprises a guide slot 82 arranged in the outer circumferential surface 80 for each of the snap ribs 66. The guide pairing formed by the snap rib 66 on the one hand and the corresponding guide slot 82 on the other hand has the effect that the respective snap rib 66 is guided in the corresponding guide slot 82 during a movement of the inner circumferential surface 84 of the retaining ring 60 relative to the outer circumferential surface 80 of the bump cap 14 corresponding to the latter, so that the positions of these components relative to one another can be adjusted in a reproducible and controllable manner.

In the embodiment example, the respective snap rib 66 is arranged on the inside of the retaining ring 60 and, correspondingly, the respective guide slot 82 is arranged on the outside of the bump cap 14; alternatively, however, the snap rib 66 could also be positioned on the bump cap 14 and, correspondingly, the guide slot 82 could be positioned on the inner circumferential surface 84 of the retaining ring 60.

As can be clearly seen from the illustration in FIG. 9, the guide slot 82 comprises a first axial segment 86 designed in the manner of an axial groove and extending in an axial direction parallel to the axis of rotation of the bump cap 14. This axial segment 86 is delimited on both sides by a respective linear guide edge 88, 90, which guide the respective snap rib 66 when the retaining ring 60 is pushed onto the bump cap 14. Furthermore, a latching bead 92 is arranged in the first axial segment 86. As soon as the snap rib 66 has been pushed over the latching bead 92 when the retaining ring 60 is pushed onto the bump cap 14, the respective snap rib 66 is latched to the latching bead 92.

Furthermore, the guide slot 82 has a tangential segment 94 designed in the manner of a tangential groove and extending in a tangential direction around the axis of rotation of the bump cap 14. In a first area, the tangential segment 94 has a lower or proximal guide edge 96, above which an extension of a second axial segment 98 is formed with an open opening area into which the respective snap rib 66 can be inserted. The guide edge 96 forms a stop for the respective snap rib 66 when the retaining ring 60 is pushed onto the bump cap 14 and thus prevents a further linear push-on movement. The tangential segment 94 merges into the axial segment 86, whereby a stop 102 for the respective snap rib 66 is formed in the transition area 100 between tangential segment 94 and axial segment 86. This is used to limit the rotation of the retaining ring 60 relative to the bump cap 14.

In the tangential segment 94 of the guide slot 82, there is also a detent tooth 104 with a beveled stop surface 106 for the snap rib 66.

As can also be clearly seen from the illustration of the bump cap 14 in FIG. 9 and the illustration of the bump cap 14 with inserted closure plug 12 and pushed-on retaining ring 60 in perspective top view in FIG. 10, a retaining collar 110 is formed on the bump cap 14 surrounding the central opening 32 in the annular lid 34. This is provided and designed so that the closure cap 20 can be attached to it via its retaining ring 70. In order to make this possible, a type of bayonet lock is also provided—in a manner analogous to the intended connection of the retaining ring 60 to the bump cap 14—in which cams arranged on the inside of the retaining ring 70 engage in an associated guide slot 112 formed on the outside of the retaining collar 110.

The medication container together with its closure system is designed to be pre-assembled after the production of the individual parts and their sterilization in such a way that the complete system can be fed into an automated filling process. In the first phase, after production and sterilization of the components, pre-assembly takes place, in which the closure system 10 and all its components are pre-assembled ready for use. It can then be delivered to a suitable filling device where it is automatically filled with the active ingredient.

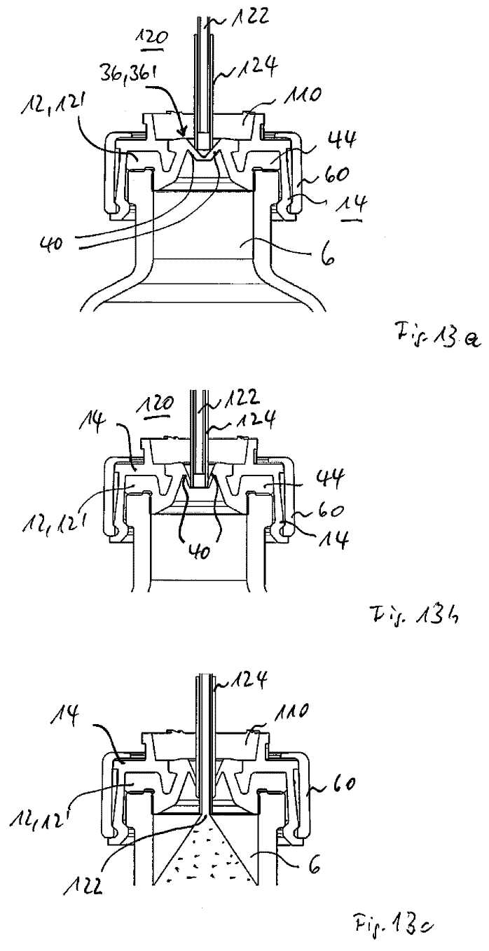

The attachment of the closure system 10 to the medication container 1 in the sense of said pre-assembly is shown in FIG. 11 by means of a sequence of steps. In a first step, shown in FIG. 11a, the provided retaining ring 60 is first positioned with its snap ribs 66 above the opening regions of the respective second axial segments 98 on the pre-assembled bump cap 14, which is already provided with the closure plug 12, 12′ comprising the closure body 36, 36′. The retaining ring 60 is then pressed linearly downwards onto the bump cap 14 and thus pushed onto it. In the process, the snap ribs 66 dip into the second axial segment 98 of the respectively assigned guide slot 82 until they abut against the guide edge 96. During pre-assembly, the respective snap rib 66 is thus initially inserted into the tangential segment 94 of its associated guide slot 82.

It is then intended to rotate the retaining ring 60 relative to the bump cap 14. This twisting causes the respective snap rib 66 to be guided in the respective tangential segment 94 until it strikes the stop 102. This ends the rotation. During rotation, the snap rib 66 is also moved over the detent tooth 104 arranged in the tangential segment 94, which is possible in this direction of rotation due to the bevel of the stop surface 106. Due to the asymmetrical contour of the detent tooth 104, however, backward rotation is then no longer possible, so that the retaining ring 60 is then also rotationally secured with respect to the bump cap 14. The retaining ring 60 is thus fixed both axially and rotationally in a defined and reproducible position on the bump cap 14.

In this position, the snap rib 66 is then also located in the transition area 100 from the tangential segment 94 of the guide slot 82 to its first axial segment 86. From this position, the retaining ring 60 can later be pushed further onto the snap-on cap 14 in the axial direction. The components are in secure engagement with each other, so that the closure system 10 pre-assembled in this way is particularly suitable for automated further processing, even under high stress and in large quantities. This corresponds to the state shown in FIG. 11a.

The bump cap 14 is then placed on the mouth area of the medication container 1 and pressed onto it until the latching elements 30 grip the outer bead behind and latch onto it. The closure plug 12, 12′ with its radial sealing element 50 penetrates into the container opening 6 until the sealing plate 44 rests with its outer edge on the mouth opening 18 of the container 1 and then, with slight deformation of the sealing plate 44 seen in the longitudinal direction of the container opening 6, the latching elements 30 of the bump cap 14 engage below the outer bead 24. The retaining ring 60 is then displaced downwards so that its cylindrical outer surface engages around the outside of the bump cap 14. This locks the latching elements 30 in their position, and the medication container 1 in the position shown in FIG. 11b is securely connected to the bump cap 14 and the closure plug 12, 12′ located in it, whereby the retaining collar 110 formed on the bump cap 14 protrudes “upwards” out of the retaining ring 60 and is therefore freely accessible. In this position shown in FIG. 11b, the medication container 1 is now ready for the temporary attachment of the closure cap 20 together with the tamper-evident closure 22 and sealing membrane 72 to the retaining collar 110.

Such a temporary attachment can be significant in an independently inventive embodiment insofar as the closure cap 20 is initially to be removed again after the pre-assembled medication container 1 has been delivered to a filling system, so that the closure body 36, 36′ designed as a valve is exposed and thus accessible and allows the medication container 1 to be filled.

For temporary attachment, the retaining ring 70 of the closure cap 20 is placed on the retaining collar 110 of the bump cap 14 in such a way that the locking pins mounted on its inner side each engage in one of the associated guide slots 112 on the retaining collar 110. Then, by suitably twisting the retaining ring 70 relative to the retaining collar 110 and the corresponding guide pairing in the respective guide slot 112, the closure cap 20 can be detachably fixed to the retaining collar 110 in such a way that the sealing membrane 72 rests against the top of the closure plug 12, 12′. The latter is thus protected from contamination by particles, aerosols and the like even in the pre-assembled state, so that, in particular when using suitable outer packaging, for example, transportation of the pre-assembled system from the production site to a filling site is possible while maintaining sterile conditions throughout. The medication container 1 pre-assembled in this way is shown in FIG. 11c.

Subsequently, the pre-assembled medication container 1 can be provided in a filling device for filling with the active ingredient. According to one aspect of the present invention, this can be done while maintaining sterile conditions due to the configuration of the closure system 10, without the need to re-sterilize the medication container 1 at the filling location. The filling of the medication container 1 is shown in FIG. 12 using a sequence of steps. As a starting point, FIG. 12a shows the pre-assembled container 1 after it has been brought to the filling location, but otherwise in the same condition as in FIG. 11c.

In a first step, in preparation for filling, the closure cap 20 is detached from the bump cap 14 by “turning it backwards”, i.e. turning its retaining ring 70 relative to the retaining collar 110 in the opposite direction to the pre-assembly described above. This produces the state shown in FIG. 12b, in which the sealing membrane 72 has been lifted off the closure plug 12, 12′, so that the closure body 36, 36′, which is designed as a valve, is exposed and can therefore be accessed. This is preferably done under sterile or aseptic conditions within the filling system.

The medication container 1 is now ready to be filled with active substance. For this purpose, in accordance with an aspect regarded as independently inventive, a filling needle system 120 of an injector unit connected to a storage container for the substance to be filled is positioned within the filling device, which is not shown, at or near the closure body 36, 36′ designed as a valve, as shown in FIG. 12c.

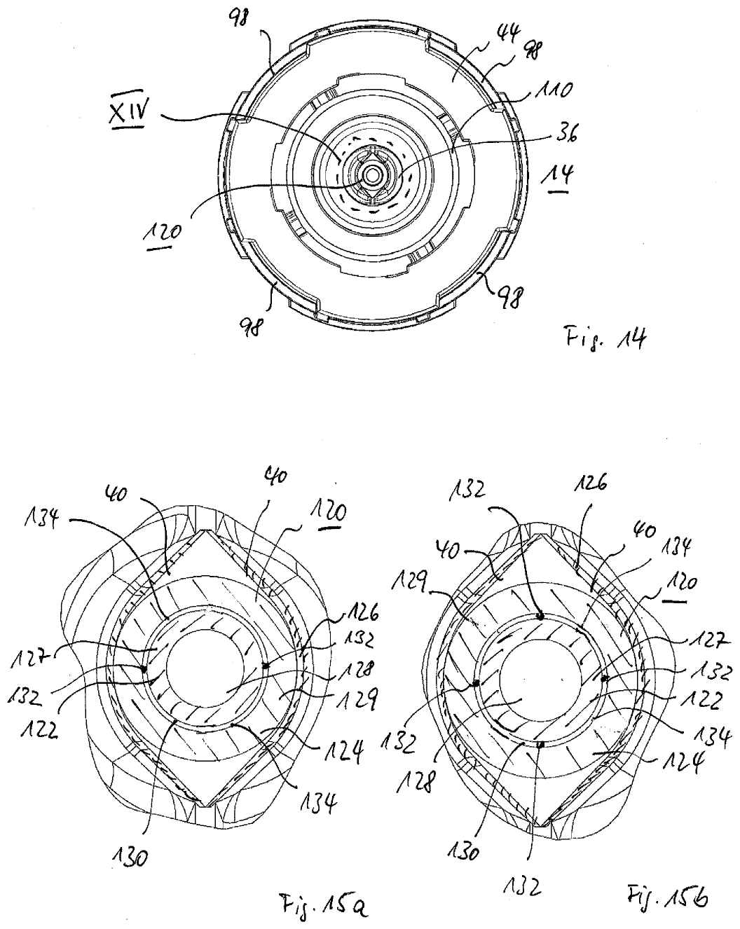

According to an aspect considered to be independently inventive, the filling needle system 120 comprises the actual filling needle 122, which is guided in an outer protective tube 124. To fill the medication container 1 with the active ingredient, the filling needle 122 is guided through the closure body 36, 36′, which is designed as a valve, and into the interior 4 of the medicament container. To illustrate the mode of operation, an enlarged view of a sequence of this insertion process is shown in FIG. 13.

Starting from the state shown in FIG. 12c, the protective tube 124 of the filling needle system 120 together with the actual filling needle 122 guided therein is first placed on the valve flanks 40 of the duckbill valve formed in the closure body 36, 36′; this position is shown in FIG. 13a. Subsequently, the protective tube 124 together with the filling needle 122 guided therein is pushed further into the closure body 36, 36′, whereby it displaces or displaces the valve flanks 40 outwards due to the design as a duckbill valve, without a diaphragm or the like having to be pierced. When the protective tube 124 is inserted into the beak area of the valve, the flank surfaces 40 forming the valve and running towards each other are thus pushed apart, whereby the valve opens. This state is shown in FIG. 13b.

The actual filling needle 122 can then be pushed forward within the protective tube 124, whereby it is pushed through the already opened gap between the valve flanks 40. Contact of the filling needle 122 with exposed surfaces such as the upper sides of the valve flanks 40 can be largely avoided due to the use of the protective tube 124, so that the entry of contamination or impurities or other particles can be kept particularly low. Such a filling concept therefore also fulfills the highest requirements with regard to maintaining sterility; it is therefore particularly suitable for filling processes under aseptic conditions. The system during filling is shown in FIG. 13c and analogously in FIG. 12d.

When the needle system 120 is pulled out after filling with the medicament or active ingredient, the flank surfaces 40 of the closure body 36, 36′ then largely close again automatically due to the elastic restoring forces in the valve body. As a result, the overall risk of contamination, external particles or the like being introduced is kept particularly low, so that filling is particularly reliable with such a closure system even under high purity requirements, in particular under sterility conditions.

After removal of the filling needle 122 from the closure body 36, 36′, the closure cap 20 is replaced, this time permanently, so that it would not be possible to open the container 1 again without destroying it, as is the case after the pre-assembly described above. The locking of the retaining ring 70 of the closure cap 20 on the retaining collar 110 of the bump cap 14 is designed in the same way as the locking of the retaining ring 60 on the bump cap 14. For this purpose, the retaining ring 70 is rotated relative to the retaining collar 110, whereby the locking pins arranged on the inside of its cylindrical outer surface are guided in the respective guide slot 112 assigned to them on the outside of the retaining collar 110. As a result of this twisting, the respective locking pin is moved over a detent tooth with a beveled front surface arranged in the respective guide slot 112, which is possible in this direction of twisting due to the bevel of the front surface. Due to the asymmetrical contour of the detent tooth, however, backward rotation is then no longer possible, so that the retaining ring 70 is then also secured against rotation with respect to the retaining collar 110 of the bump cap 14. The closure cap 20 is thus fixed to the bump cap 14 in a tamper-proof manner, and access to the container interior 4 is only possible by opening the tamper-evident closure, i.e. by tearing off the sealing plate 68 from the retaining ring 70 and then piercing the sealing membrane 72.

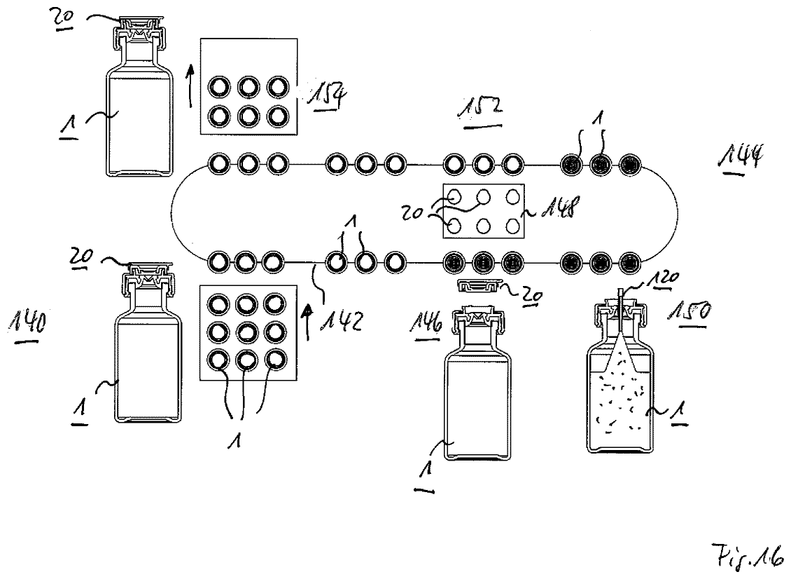

As can be seen from the illustrations in FIG. 13, the filling needle system 120 thus forms a tube-in-tube system with which different media channels can be formed as required and depending on the active ingredient to be filled, in a design that is regarded as independently inventive. By way of explanation, FIG. 14 shows the bump cap 14 with inserted filling needle system 120; FIG. 15 shows two different versions of the central area of the bulging cap in enlarged cross-section. Clearly recognizable in these illustrations are the valve flanks 40, which are spread apart when the filling needle system 120 is inserted into the duckbill valve formed by the closure body 36, and which form a lip-like circumferential contour 126 in this inserted state; when the filling needle system 120 is removed, this contour 126 closes again due to the restoring forces in the elastic material forming the valve, forming the contact line 42 described above.

As can be seen from the enlarged cross-sectional views in FIG. 15, the filling needle 122 forms an inner media channel 128 enclosed by its needle jacket 127, which is intended for use as a medication channel and thus for transporting the medication or active ingredient into the container interior 4. As described above, the filling needle 122 is surrounded by the protective tube 124. An annular gap 130 is formed between its needle jacket 129 and the outside of the needle jacket 127 of the filling needle 122. In an independently inventive embodiment, it is now envisaged that this annular gap 130 can also be used as a media channel, in particular for transporting gases into and out of the container interior 4. On the one hand, it is necessary to allow the air present in the container interior 4 (or any protective or inert gas present there) to escape when filling the medication container 1 with active ingredient; the annular gap 130 can be used as a media channel for this so-called “venting” Furthermore, once the active ingredient has been introduced into the container interior 4, the introduction of protective or inert gas, such as nitrogen, into the container interior 4 can also be provided or favored. Such a protective or inert gas can, for example, serve as a gas cushion to minimize or completely prevent contact of the active ingredient with atmospheric oxygen and the resulting chemical reactions, thus significantly improving the shelf life of the active ingredient.

According to this aspect of the disclosure, the annular gap 130 is thus used as a media channel for venting and/or for introducing protective or other functional gas into the container interior 4. For this purpose, the annular gap is subdivided into a plurality of gap segments 134 by means of a number of partition walls 132 arranged therein, each of which can be used as a media channel for an associated medium. The type and number of these partition walls 132 and the gap segments 134 formed thereby are preferably suitably selected with regard to the details of the intended filling process, for example the number of ventings required and/or the intended gas components. FIG. 15a shows an embodiment with two gap segments 134 in the annular gap 130, whereas FIG. 15b shows a variant with four gap segments 134.

The simultaneous filling of a plurality of such medication containers 1 can be carried out automatically in a filling process, as shown schematically in FIG. 16. In addition to the respective schematically indicated process steps, one of the medication containers 1 is shown in its current state for illustration purposes.

During filling, in a first step 140, a plurality of medication containers 1 with pre-assembled closure system are initially fed in a common arrangement (“nesting”, shown in plan view in the diagram) to a transport system 142 of the filling device 144 and separated there in the sense that they are now conveyed sequentially one behind the other in the transport system. Via the transport system 142, they are conveyed sequentially to a unit in which, in a step 146, the closure cap 20 is removed by the “reverse rotation” described above and fed to intermediate storage in an intermediate interim storage 148. Then, in a filling step 150, the actual filling takes place according to the concept described above. Finally, the closure caps 20 are removed again from the interim storage 148 and, in a step 152, applied again to the respective, now filled medication container 1. In this step, the application is now latching, so that the respective closure cap 20 can no longer be removed from the respective medication container 1 in a non-destructive manner. Subsequently, in a step 154, the now filled and tamper-proof sealed medication containers 1 are again combined into several bundles or pallets and conveyed out of the filling device 144 in these units.

### LIST OF REFERENCE SYMBOLS

1 Medication container
2 Container wall
4 Interior
6 Container opening
10 Closure system
12 Closure plug
14 Bump cap
16 Fixing cap
18 Mouth opening
20 Closure cap
22 Tamper-evident closure
24 Outer bead
30 Latching elements
32 Opening
34 Annular lid
36,36′ Closure body
38 Groove
40 Valve flank
42 Contact line
44 Sealing plate
46 Mouth edge
50 Radial sealing element
52 Recess
54 Reinforcing ring
60 Retaining ring
62 Opening
64 Outer surface
66 Snap ribs
68 Sealing plate
70 Retaining ring
72 Sealing membrane
74 Fixing points
80 Circumferential surface
82 Guide slot
84 Circumferential surface
86 Axial segment
88, 90 Guide edge
92 Latching bead
94 Tangential segment
96 Guide edge
98 Second axial segment
100 Transition area
102 Stop
104 Detent tooth
106 Stop surface
110 Retaining collar
112 Guide slot
120 Filling needle system
122 Filling needle
124 Protective tube
126 Contour
127 Needle jacket
128 Media channel
129 Needle jacket
130 Annular gap
132 Partition wall
134 Gap segment
140 Step
142 Transport system
144 Filling device
146 Step
148 Interim storage
150 Filling step
152, 154 Step

1. A closure system for a medication container, an interior of which is accessible via a container opening configured in the form of a bottle mouth, comprising:
a bump cap with an annular lid having a central opening and on the outer circumference of which a number of latching elements are arranged which can be brought into engagement with an outer bead-mounted circumferentially on the container opening; and
a one-piece closure plug including a closure body that completely fills the central opening of the annular lid and can be brought into engagement therewith in a latching manner, wherein the closure body is configured as a duckbill valve.

2. The closure system according to claim 1, further comprising a radial sealing element integrally formed radially circumferentially on the closure body of the closure plug, the radial sealing element having a cross-section configured to a clear width of the container opening and is slightly larger than the clear width of the container opening with regard to deformability of material of the closure plug.

3. The closure system according to claim 1, further comprising a retaining ring which can be slid on to the bump cap and which, in a position completely slid on to the bump cap, can be latchably fixed to the bump cap by a plurality of snap ribs.

4. The closure system according to claim 3, wherein each snap rib is guided in a corresponding guide slot during a movement of an inner circumferential surface of the retaining ring relative to an outer circumferential surface of the bump cap.

5. The closure system according to claim 1, further comprising a retaining collar formed on the bump cap surrounding the central opening for attachment of an associated closure cap.

6. The closure system according to claim 5, wherein the closure cap includes a retaining ring having a pierceable sealing membrane that rests on the closure body in an assembled state when the closure system is assembled on the medication container.

7. The closure system according to claim 6, wherein a removable sealing plate is attached to the retaining ring and is configured to be torn off the retaining ring to form a tamper-evident closure.

8. The closure system according to claim 5, further comprising a plurality of guide slots formed on an outside of the retaining collar of the bump cap, in each of which a corresponding locking pin arranged on an inside of the retaining ring of the closure cap is guided.

9. A medicament container, having an interior of which is accessible via a container opening configured in the form of a bottle mouth, which is provided with the closure system according to claim 1.

10. A filling device for the medicament container according to claim 9, comprising an injector unit connected to a storage container for a substance to be filled, the injector unit including a filling needle guided in an outer protective tube.
